(19) United States
(12) Patent Application Publication
(10) Pub. No.: US 2013/0273187 A1
(43) Pub. Date: Oct. 17, 2013

(54) METHOD OF TREATING ACNE

(71) Applicant: Viktor Vladimirovich Redkin, Moscow (RU)

(72) Inventor: Viktor Vladimirovich Redkin, Moscow (RU)

(21) Appl. No.: 13/976,516

(22) PCT Filed: Dec. 19, 2011
(86) PCT No.: PCT/RU2011/001004

§ 371 Date: Jun. 27, 2013

(30) Foreign Application Priority Data
Dec. 27, 2010 (RU) 2010153384

(51) Int. Cl. A61K 9/00; A61K 35/74

(52) U.S. Cl. CPC A61K 9/0014; A61K 35/74
USPC 424/780

(57) ABSTRACT

The invention relates to methods of treating acne, which methods comprise topical administering to a skin of a subject in need thereof an effective amount of a lysate of Actinomyces, preferably strains R/3.88, L/1.89, or mixture thereof.

### TECHNICAL FIELD

The present invention relates to medicine, particularly to a method of treating acne with lysate of Actinomyces.

### BACKGROUND ART

Acne is a widely distributed disease of a human skin. Actinomyces is a class of bacteria. USSR Patent No. 81396 (priority date of Sep. 27, 1948) describes a method of preparing lysate of Actinomyces and the use of the lysate for diagnostics and treating actinomycosis in humans with subcutaneous and intramuscular injections. USSR Patent No. 584952 (priority date of Jul. 7, 1988) describes a method of preparing lysate of Actinomyces purified from high molecular weight fractions 15000-25000 Daltons. Guideline for physicians “The use of Actinolysate in clinical practice” (Moscow, 2005) describes the use of injections of lysate of Actinomyces (Actinolysate) for the treatment of skin diseases, including abrasions, abscesses, cellulitis, acne, purulent fistulas, sores, pressure sores, hidradenitis, the lungs and liver abscess, peritonitis, cystitis, prostatitis, bacterial vaginitis, purulent mastitis, for the treatment of actinomycosis cervical-facial, skin and soft tissues, bone structure, internal organs, adrectal and genital areas, and for the treatment of purulent sinusitis, a bacterial tonsillitis, purulent otitis media, lymphadenopathy, for the treatment of odontogenic inflammation, chronic osteomyelitis, purulent bursitis purulent bacterial infections. Thus, the treatment of acne with lysates of Actinomyces is known from the art. However, only injections of lysates of Actinomyces were used for the treatment of acne and such lysates have never been used for the treatment of acne under topical route of administration.

Lysates of Actinomyces are multicomponent compositions containing hydrophilic substances of different molecular mass, including compounds of high molecular weight (>1000 Daltons). As a it is generally accepted that human skin represents an effective barrier against absorption of hydrophilic compounds, especially with high molecular weight, it is obvious for a person ordinary skilled in the art that components of lysates of Actinomyces cannot be absorbed well by skin in therapeutically effective concentrations to provide a therapeutic effect under topical application onto skin surfaces.

I discovered that bioavailability of lysates of Actinomyces under topical route of administration is increased, when such lysates are used in formulations, which provides prolong contact with a skin for times sufficient for delivering active components of the lysates through the skin. Additively, the bioavailability of such lysates is increased upon use of substances, which improve absorption components of lysates through the skin. Topical route of administration of lysates is superior to injections for treating diseases of skin and nails. Injections induce undesirable systemic side effects, which can be avoided under topical administration. Also, injections are non-comfortable for subjects and may provide illness, irritation, and inflammation in the place of injection. Topical route of lysate administration provides lowering dose of lysates, as it provides therapeutically effective local concentrations of components of lysates at the place of acne and, thus, may decrease side effects arising from high doses of lysates under injections. Therefore, there is a need in the topical formulations of lysates of Actinomyces for the treatment of acne

It is an object of present invention to provide methods of treating acne, which comprises a step of topical administering an effective amount of a lysate of Actinomyces to a skin of a subject in need thereof.

### DISCLOSURE OF INVENTION

The present invention provides a method of treating acne, which comprises a step of topical administering to a skin of a subject in need thereof an effective amount of a lysate of Actinomyces.

The term “lysate” means a sterile filtrate of a liquid medium, obtained due to lysis of Actinomyces previously grown in culture. The lysate is prepared by methods well-known from the art. For example, cultivation of Actinomyces for 1.5 to 2 months at 37° C. results in spontaneous lysis of culture, the cultural medium is filtered under sterile conditions, and the lysate is obtained upon drying the filtrate.

The term “topical” means that a formulation comprising lysate of actinomyces is applied onto a skin of a human in need thereof.

The lysate of Actinomyces can be used for the treatment of acne and related conditions, such as comedones, polymorphous acne, acne rosacea, nodular acne, senile acne, secondary acne such as solar acne and acne arising from the use of drugs. Preferably, the effective amount of lysate of Actinomyces for the use in the method of present invention is from 0.0001 to 10 wt. %, more preferably, from 0.01 to 0.05 wt. %.

Preferably, Actinomyces of the present invention is selected from the group consisting of strains R/3.88, L/1.89, or mixture thereof.

According to the present invention, lysate of Actinomyces can be applied onto a skin as a component of topical formulation. The formulation can contain additives. Non-exclusive examples of such additives are gel forming agents, preservatives, dyes, fragrances, pH regulators, osmolality regulators, antioxidants. Preferably, the amount of auxiliary components in the pharmaceutical composition of the present invention is 0.0001 to 20.00 wt. %, more preferably 0.1-5.0 wt. %. Preferably, the formulation of the present invention has a pH of 4.5-7.5. The formulation can be prepared in different forms, which include, but are not limited to, solutions, gels, emulsions, ointments, patches, or forms that allow a controlled release of the active components of the lysate. The formulation can be prepared by methods well-known from the art. For example, lysate of Actinomyces, benzalkonium chloride as preservative, and carboxyethylcellulose as gel forming agent are dissolved in purified water as excipient and resulted gel is packed in unit dosage forms.

The following examples are presented to demonstrate the invention. The examples are illustrative only and are not intended to limit the scope of invention in any way.

### EXAMPLE 1

Table 1 demonstrates topical gel formulation for the treatment of acne, which formulation contains lysate of Actinomyces.

TABLE 1

| Ingredients | Content, wt. % |
|---|---|
| Lysate of Actinomyces | 0.0001-10 |
| Carboxyethylcellulose | 2 |
| Benzalkonium chloride | 0.2 |
| EDTA | 0.1 |
| Purified water | Up to 100% |

Preparation of the formulation: lysate of Actinomyces, carboxyethylcellulose as a gelling agent, benzalkonium chloride, EDTA was added to water and mixed. The resulting product is placed in the tube. Dosage: 1 ml of the product is applied to the skin of a subject with acne, once a day for several days.

### EXAMPLE 2

Three women of 18, 25 and 21 years of age, applied the formulation of Table 1 to a face skin with signs of acne once a day for four weeks. As results, the appearance of facial skin and function of sebaceous gland were improved and signs of acne were diminished.

I claim:

1. A method of treating acne, which comprises a step of topical administering to a skin of a subject in need thereof an effective amount of a lysate of Actinomyces.

2. The method of claim 1, wherein said Actinomyces is selected from the group consisting of strains R/3.88, L/1.89, or mixture thereof.
